(19) United States
(12) Patent Application Publication
(10) Pub. No.: US 2002/0137795 A1
(43) Pub. Date: Sep. 26, 2002

(54) Composition containing retinol and a compound capable of screening out UVA radiation

(75) Inventors: Guenaelle Martin, Paris (FR); Philippe Touzan, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/978,027

(22) Filed: Oct. 17, 2001

(30) Foreign Application Priority Data
Oct. 17, 2000 (FR) 00 13285

(51) Int. Cl. A61K 31/185

(52) U.S. Cl. 514/553

(57) ABSTRACT

The present invention relates to a composition containing retinol and a compound capable of screening out UVA radiation corresponding to formula (I):

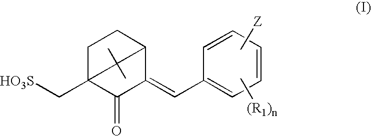

which is preferably benzene-1,4-di(3-methylidene-10-camphorsulphonic acid).

### BACKGROUND OF THE INVENTION

1. Field of the Invention

The present invention relates to novel compositions, preferably cosmetic and/or dermatological compositions, in particular compositions intended for caring for the skin, comprising, preferably in a physiologically acceptable medium, retinol and a compound capable of screening out UVA radiation. Methods of use of these compositions, and methods of preparing said compositions, also make up a part of the invention.

2. Discussion of the Background

Cosmetic and/or dermatological compositions based on retinoids have experienced significant growth in recent years, in particular for the treatment of acne and cutaneous imperfections, because of their keratolytic power, and for the prevention and treatment of certain signs of intrinsic or photoinduced cutaneous ageing, such as the formation of wrinkles and the loss of firmness and elasticity of the skin.

Among the derivatives of the retinoid family, retinol, also known under the name of vitamin A, is of very particular advantage. This is because retinol is a natural endogenous constituent of the human body which is well tolerated on application to the skin up to levels which are much higher than those of retinoic acid.

However, when it is introduced into a cosmetic or dermatological composition intended for a topical application, retinol rapidly decomposes under the effect of light, oxygen, metal ions, oxidizing agents or water or, in particular, under the effect of a rise in temperature. The thermal decomposition of retinol has formed the subject of a study published in J. Soc. Cosm. Chem., 46, 191-198 (July-August 1995).

Furthermore, the inventors have noted that the introduction of some UVA screening agents into cosmetic compositions comprising retinol led to decomposition of the retinol over time, without the reasons for this decomposition being able to be clearly identified.

This fact is particularly unfortunate as regards compositions intended for the prevention or for the treatment of cutaneous signs of photoageing, in so far as it is often advantageous to combine, in these compositions, the biological effects of retinol, in particular on the synthesis of collagen, with the effects of the sunscreen agents which absorb UVA radiation. This is because these screening agents make it possible to help the cells defend themselves against the excess photoinduced free radicals and to prevent the decomposition of collagen fibres due to UVA radiation. They thus have antiageing effects which are complementary to those of retinol.

### OBJECTS OF THE INVENTION

An object of the invention is to provide compositions, in particular cosmetic compositions, comprising both retinol and a UV screening agent, in particular a UVA screening agent, in which compositions the retinol is not decomposed by the screening agent.

### SUMMARY OF THE INVENTION

The inventors have discovered with astonishment that a given family of UVA screening agents can be used in compositions comprising retinol without leading to decomposition of the latter. These preferably cosmetic and/or dermatological compositions can thus be stored for several months without experiencing a decline in their effectiveness.

### DETAILED DESCRIPTION OF THE INVENTION

The invention compositions comprise, preferably in a physiologically acceptable medium, retinol and a compound capable of screening out UVA radiation, characterized in that the said compound corresponds to the formula (I):

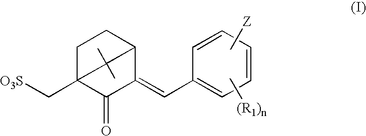

in which

Z denotes a group of formula:

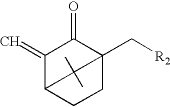

where attachment to formula (I) occurs at the vinyl methylene ═CH,

R₂ denoting —H or —SO₃H;

n denotes 0 or an integer of greater than or equal to 1 and less than or equal to 4;

R₁ represents one or more identical or different linear or branched alkyl or alkoxy radicals comprising from 1 to 4 carbon atoms,

the two methylidenecamphor radicals

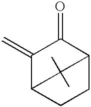

being arranged on the phenyl nucleus in a meta or para orientation with respect to one another.

Optionally, the sulphonic acid functional group of the compound of formula (I) can be entirely or partially neutralized by an alkali metal or alkaline earth metal hydroxide, ammonia or an organic base.

The compounds of formula (I) above may be obtained by or made by one of ordinary skill in this art in view of the disclosure herein and are disclosed respectively in U.S. Pat. No. 4,585,597 and Patent Applications FR 2 236 515, 2 282 426, 2 645 148, 2 430 938 and 2 592 380, all incorporated herein by reference.

A particularly preferred compound of formula (I) is benzene-1,4-di(3-methylidenecamphor-10-sulphonic acid) of formula (II):

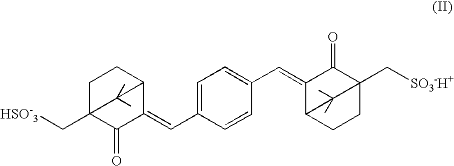

In an alternative form, use may be made, in the present invention, of one of the above compound's alkali metal, alkaline earth metal or ammonium salts or one of its salts with an organic base. It is a sunscreen capable of absorbing ultraviolet rays with a wavelength of between 280 and 400 nm, with absorption maxima of between 320 and 400 nm, in particular in the vicinity of 345 nm. This compound also has the property, when it comprises at least one at least partially unneutralized sulphonic acid functional group, of combating the intrinsic ageing of the skin, as indicated in Application EP-0 671 160, incorporated herein by reference.

For better protection with respect to UV radiation, the composition according to the invention can, in addition to the abovementioned UVA screening agent of formula (I), include at least one compound capable of screening out UVB radiation and/or at least one other compound capable of screening out UVA radiation and/or at least one optionally coated inorganic pigment.

While not being limited, mention may particularly be made, as compounds capable of screening out UVB radiation, of:

(1) salicylic acid derivatives, in particular homomenthyl salicylate and octyl salicylate;

(2) cinnamic acid derivatives, in particular 2-ethylhexyl p-methoxycinnamate, available from Givaudan under the trade name Parsol MCX;

(3) liquid β,β′-diphenylacrylate derivatives, in particular 2-ethylhexyl α-cyano-β,β′-diphenylacrylate, or octocrylene, available from BASF under the trade name Uvinul N539;

(4) p-aminobenzoic acid derivatives;

(5) 4-methylbenzylidenecamphor, available from Merck under the trade name Eusolex 6300;

(6) 2-phenylbenzimidazole-5-sulphonic acid, sold under the trade name “Eusolex 232” by Merck;

(7) 1,3,5-triazine derivatives, in particular:

2,4,6-tris[4-(ethylhexyloxycarbonyl)anilino]-1,3,5-triazine, available from BASF under the trade name Uvinul T150, and

the compound corresponding to the following formula:

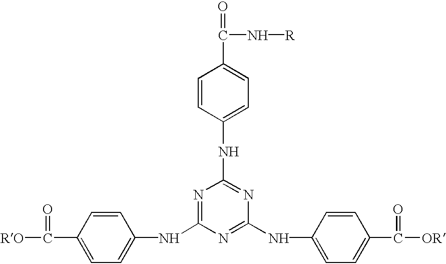

in which R′ denotes a 2-ethylhexyl radical and R denotes a tert-butyl radical, available from Sigma 3V under the trade name Uvasorb HEB;

(8) their mixtures.

While not being limited, mention may in particular be made, as other (non-formula (I)) compounds capable of screening out UVA radiation, of (1) benzophenone derivatives, for example:

2,4-dihydroxybenzophenone (benzophenone-1);

2,2′,4,4′-tetrahydroxybenzophenone (benzophenone-2);

2-hydroxy-4-methoxybenzophenone (benzophenone-3), available from BASF under the trade name Uvinul M40;

2-hydroxy-4-methoxybenzophenone-5-sulphonic acid (benzophenone-4) and its sulphonate form (benzophenone-5), available from BASF under the trade name Uvinul MS40;

2,2′-dihydroxy-4,4′-dimethoxybenzophenone (benzophenone-6);

5-chloro-2-hydroxybenzophenone (benzophenone 7);

2,2′-dihydroxy-4-methoxybenzophenone (benzophenone-8);

the disodium salt of 2,2′-dihydroxy-4,4′-dimethoxybenzophenone-5,5′-disulphonic diacid (benzophenone-9);

2-hydroxy-4-methoxy-4′-methylbenzophenone (benzophenone-10);

benzophenone-11;

2-hydroxy-4-(octyloxy)benzophenone (benzophenone-12),

benzophenone-3 and benzophenone-5 being preferred; (2) triazine derivatives and in particular 2,4-bis[4-(2-ethylhexyloxy)-2-hydroxyphenyl]-6-(4-methoxyphenyl)-1,3,5-triazine, available from Ciba-Geigy under the trade name Tinosorb S, and 2,2′-methylenebis(6-(2Hbenzotriazol-2-yl)-4-(1,1,3,3-tetramethylbutyl)phenol], available from Ciba-Geigy under the trade name Tinosorb M;

(3) their mixtures.

The term “optionally coated inorganic pigments” is understood to mean in particular coated or noncoated metal oxide nanopigments (mean size of the primary particles: generally between 5 nm and 100 nm, preferably between 10 nm and 50 nm), such as, for example, titanium oxide (amorphous or crystallized in the rutile and/or anatase form), iron oxide, zinc oxide, zirconium oxide or cerium oxide nanopigments, which are all UV photoprotective agents well known per se. Conventional coating agents are, furthermore, alumina and/or aluminium stearate. Such coated or noncoated metal oxide nanopigments are disclosed in particular in Patent Applications EP-A-0 518 772 and 15 EP-A-0 518 773, both incorporated herein by reference.

The compositions according to the invention are preferably intended for a cosmetic or dermatological use, advantageously a cosmetic use. It is preferably intended for topical application and therefore generally preferably comprises a physiologically acceptable medium, that is to say a medium which is compatible with the skin.

The composition according to the invention can generally comprise an amount of retinol effective in producing the desired effect, for example of between 0.01 and 0.2% by weight and preferably of between 0.01 and 0.15% by weight, with respect to the total weight of the composition. It additionally includes an amount of UVA screening agent of formula (I) sufficient to confer the desired Sun Protection Factor on it, for example from 0.5 to 5% by weight and preferably from 0.7 to 3% by weight of UVA screening agent of formula (I), with respect to the total weight of the composition.

This composition can be provided in any form, and is not limited. Pharmaceutical dosage and forms normally used in the cosmetic and dermatological fields are included, and it can in particular be in the form of an optionally gelled aqueous solution, of a dispersion of the lotion type, optionally with two phases, of an emulsion obtained by dispersion of a fatty phase in an aqueous phase (O/W) or vice versa (W/O), of a triple emulsion (W/O/W or O/W/O) or of a vesicular dispersion of ionic and/or nonionic type. These forms may be prepared according to conventional methods. Preferably, the composition according to the invention is in the form of an oil-in-water emulsion.

The composition of the invention can be more or less fluid and can have the appearance of a white or colored cream, of an ointment, of a milk, of a lotion, of a serum, of a paste or of a foam. It can optionally be applied to the skin in the aerosol form. It can also be provided in the solid form, in particular in the form of a stick for the lips. It can be used as a care product and/or as a make-up product for the skin.

In a known way, the composition of the invention can also comprise adjuvants which are conventional in the cosmetics field, such as hydrophilic or lipophilic gelling agents, hydrophilic or lipophilic active principles, preservatives, antioxidants, solvents, fragrances, fillers, pigments, odour absorbers and colouring materials. The amounts of these various adjuvants are those conventionally used in the field under consideration, for example from 0.01 to 20% of the total weight of the composition. These adjuvants, depending upon their nature, can be introduced into the fatty phase, into the aqueous phase, into the lipid vesicles and/or into the nanoparticles. In any event, these adjuvants, and their proportions, will be chosen so as not to harm the desired properties of the combination according to the invention.

When the composition of the invention is an emulsion, the proportion of the fatty phase preferably can range from 5 to 80% by weight and more preferably from 5 to 50% by weight with respect to the total weight of the composition. The oils, emulsifiers and coemulsifiers used in the composition in the emulsion form can be chosen from those conventionally used in the field under consideration. The emulsifier and the coemulsifier may be present in the composition in a proportion ranging from 0.3 to 30% by weight and preferably from 0.5 to 20% by weight with respect to the total weight of the composition.

While not limited, mention may be made, as oils which can be used in the invention, of mineral oils (liquid petrolatum), oils of vegetable origin (avocado oil, soybean oil), oils of animal origin (lanolin), synthetic oils (perhydrosqualene), silicone oils (cyclomethicone) and fluorinated oils (perfluoropolyethers). Use may also be made, as fatty substances, of fatty alcohols (cetyl alcohol), fatty acids or waxes (carnauba wax, ozokerite).

While not limited, mention may be made, as emulsifiers and coemulsifiers which can be used in the invention, of, for example, polyethylene glycol fatty acid esters, such as PEG-100 stearate, and glycerol fatty acid esters, such as glyceryl stearate.

While not limited, mention may in particular be made, as hydrophilic gelling agents, of carboxyvinyl polymers (carbomer), acrylic copolymers, such as acrylate/alkylacrylate copolymers, polyacrylamides, polysaccharides, natural gums and clays, and mention may be made, as lipophilic gelling agents, of modified clays, such as bentones, metal salts of fatty acids, hydrophobic silica and polyethylenes.

The present invention also relates to the cosmetic uses of the composition described above, in particular for preventing or treating signs of intrinsic or photoinduced cutaneous ageing. Also, methods of preparing the invention compositions are included where retinol and at least one compound of Formula (I) are mixed, added together, added one to the other, etc, hereinafter referred to as “contacted” or “contacting.”

The invention will be better understood and its advantages will more clearly emerge in the light of the following examples, which are given by way of illustration and without limitation.

### EXAMPLES

### Example 1

### Cosmetic Composition

| Phase A | |
|---|---|
| Glyceryl stearate and PEG-100 stearate | 2.1% |
| Polysorbate 60 | 0.9% |
| Cetyl alcohol | 2.6% |
| Hydrogenated polyisobutene | 12% |
| Hexyldecanol | 8% |
| BHT | 0.1% |
| Preservative | 0.15% |
| Phase B | |
| Water | q.s, for |
| | 100% |
| Glycerol | 3% |
| Preservative | 0.55% |
| Pentasodium salt of ethylenediamine- | 0.07% |
| tetramethylenephosphonic acid | |
| Phase C | |
| Xanthan gum | 0.1% |
| Carbomer | 0.4% |
| Phase D | |
| Water | 5% |
| Triethanolamine | 0.38% |
| Phase E | |
| Benzene-1,4-di(3-methylidene-10-camphor | 1% |
| sulphonic acid) | |
| Triethanolamine | 0.19% |
| Phase F | |
| Retinol | 0.1 |

The above composition can be prepared in the following way.

Phases A and B are heated separately with stirring at 75° C. until completely dissolved. Phases D and E are prepared separately with stirring at ambient temperature. Phase A is subsequently decanted into phase B with stirring at 75° C. over 5-10 min and then the combined mixture is cooled to 50° C. The constituents of phase C are subsequently introduced into the mixture of phases A and B with stirring at 50° C. and, after complete homogenization, phases D and E are successively added with stirring. The mixture is cooled to ambient temperature. After rendering inert with nitrogen, phase F is introduced into the vessel with stirring.

This composition can be used daily as a day cream for preventing and combating wrinkles and for toning up the skin.

### Example 2

### Demonstration of the Stability of the Retinol

The stability of the retinol was evaluated in the three compositions A to C below:

Composition A: Composition of Example 1 without phase E

Composition B: Composition of Example 1

Composition C: Composition of Example 1 without phase E and with 1% of 4-tert-butyl-4′-14 methoxydibenzoylmethane (sold under the name Parsol 1789 by Givaudan) in phase A.

The amounts by weight of retinol respectively present in compositions A to C above were determined, first at ambient temperature, immediately after preparation (To) of these compositions, and, secondly, after storage for two months at 45° C. (T2m).

These measurements were made by High Pressure Liquid Chromatography using, as standard, a 16 μg/ml solution of retinol in THF prepared from a sample of Retinol 10S sold by BASF in the form of a 10% oily solution of retinol.

The results obtained are as follows:

| | To | T2m |
|---|---|---|
| Composition A | 0.108% | 0.102% |
| Composition B | 0.108% | 0.097% |
| Composition C | 0.105% | 0.071% |

Taking into consideration the accuracy of the method of quantitative determination used, it may be deduced therefrom that composition B, comprising benzene-1,4-di(3-methylidene-10-camphorsulphonic acid) as UVA screening agent, is as stable as composition A, not comprising UV screening agent, and markedly more stable than composition C, comprising another UVA screening agent.

French patent application 0013285 filed Oct. 17, 2000 is incorporated herein by reference.

1. A composition comprising retinol and a compound of formula (I): 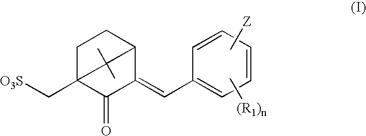
in which
Z denotes a group of the formula: 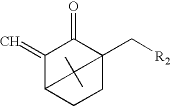
R2 denoting —H or —SO₃H;
denotes 0 or an integer of greater than or equal to 1 and less than or equal to 4;
R₁ represents one or more identical or different and linear or branched alkyl or alkoxy radicals comprising from 1 to 4 carbon atoms,
the two methylidenecamphor radicals being arranged on the phenyl nucleus in a meta or para orientation with respect to one another, each sulphonic acid functional group of which can optionally be entirely or partially neutralized by an alkali metal or alkaline earth metal hydroxide, ammonia or an organic base.

2. The composition according to claim 1, wherein the compound of formula (I) is benzene-1,4-di(3-methylidene-10-camphorsulphonic acid) or one of its alkali metal, alkaline earth metal or ammonium salts or one of its salts with an organic base.

3. The composition according to claim 1, comprising from 0.01 to 0.15% by weight of retinol, with respect to the total weight of the composition.

4. The composition according to claim 1, comprising from 0.7 to 3% by weight of said compound of formula (I), with respect to the total weight of the composition.

5. The composition according to claim 1, characterized in that it is in the form of an oil-in-water emulsion.

6. The composition according to claim 1, further comprising at least one compound capable of screening out UVB radiation and/or at least one non-formula I compound capable of screening out UVA radiation and/or at least one optionally coated inorganic pigment.

7. The composition according to claim 1, further comprising a compound capable of screening out UVB radiation selected from the group consisting of:
(1) salicylic acid derivatives,
(2) cinnamic acid derivatives
(3) liquid β,β′-diphenylacrylate derivatives
(4) p-aminobenzoic acid derivatives;
(5) 4-methylbenzylidenecamphor;
(6) 2-phenylbenzimidazole-5-sulphonic acid;
(7) 1,3,5-triazine derivatives, and (8) mixtures thereof.

8. The composition according to claim 1, further comprising a compound capable of screening out UVB radiation selected from the group consisting of:
(1) homomenthyl salicylate and octyl salicylate,
(2) 2-ethylhexyl-p-methoxycinnamate,
(3) 2-ethylhexyl-α-cyano-β,β′-diphenylacrylate, or octocrylene,
(4) 4-methylbenzylidenecamphor,
(5) 2-phenylbenzimidazole-5-sulphonic acid,
(6) 2,4,6-tris[4-(ethylhexyloxycarbonyl)anilino]1,3,5-triazine, and
(7) a compound corresponding to the following formula: 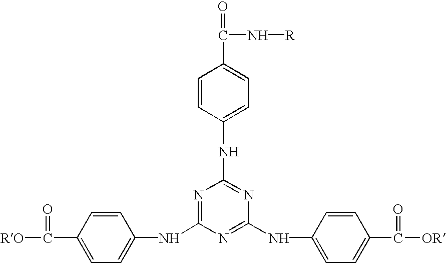
in which R′ denotes a 2-ethylhexyl radical and R denotes a tert-butyl radical.

9. The composition according to claim 1, further comprising a compound capable of screening out UVA radiation selected from the group consisting of:
(1) benzophenone derivatives,
(2) triazine derivatives, and
(3) mixtures thereof.

10. The composition according to claim 1, further comprising a compound capable of screening out UVA radiation selected from the group consisting of:
2-hydroxy-4-methoxybenzophenone (benzophenone-3), 2-hydroxy-4-methoxybenzophenone-5-sulphonic acid,
2,4-bis[4-(2-ethylhexyloxy)-2-hydroxyphenyl]-6-(4methoxyphenyl)-1,3,5-triazine, and 2,2′-methylenebis[6(2H-benzotriazol-2-yl)-4-(1,1,3,3 -tetramethylbutyl)phenol].

11. The composition according to claim 1, further comprising an inorganic pigment selected from the group consisting of from titanium oxide, iron oxide, zinc oxide, zirconium oxide and cerium oxide nanopigments, optionally coated with alumina and/or with aluminium stearate.

12. A method for preventing or treating signs of intrinsic or photoinduced cutaneous ageing comprising applying the composition of claim 1 to the skin, lips or scalp.

13. A method for preparing the composition of claim 1, comprising contacting said retinol and said compound of formula (I).
